(19) United States
(12) Patent Application Publication
(10) Pub. No.: US 2009/0170061 A1
(43) Pub. Date: Jul. 2, 2009

(54) DEVICE COMPRISING A SAMPLE OF RECONSTRUCTED TISSUE AND A DETECTION SYSTEM

(76) Inventor: Jean-Luc Leveque, Paris (FR)

Correspondence Address: O'BRIEN JONES, PLLC, 8200 Greensboro Drive, Suite 1020A, McLean, VA, 22102, US

(21) Appl. No.: 11/719,291

(22) PCT Filed: Nov. 15, 2005
(86) PCT No.: PCT/IB2005/053770

§ 371 Date: Feb. 9, 2009

Related U.S. Application Data
(60) Provisional application No. 60/631,191, filed on Nov. 29, 2004.

(30) Foreign Application Priority Data
Nov. 15, 2004 (FR) 04 52626

(51) Int. Cl. C12Q 3/00; C12M 3/00; C12N 5/06

(52) U.S. Cl. 435/3; 435/284.1

(57) ABSTRACT

The present invention relates to a device (1) comprising: a sample of reconstructed tissue (4); and a detection system (20), (30) that is at least partially in contact with the tissue sample and/or with a device serving to culture said sample, said detection system making it possible to measure the influence of a physical, biological, and/or chemical stress on at least a fraction of the sample, or vice-versa.

The present invention relates to devices associating a sample of reconstructed tissue, e.g. of reconstructed skin, with a detection system.

Several types of reconstructed skin are known that are characterized by the similarity of their epidermis with natural skin.

A first epidermis model contains keratinocytes exclusively. The use of that model makes it possible, amongst other things, to simulate aging of the skin.

A second epidermis model also contains melanocytes. That model is pigmented and makes it possible to understand better the phenomenon of melanogenesis.

A third epidermis model contains keratinocytes, melanocytes, and Langerhans cells. That epidermis model makes it possible to understand the phenomenon of photoimmunosuppression, or the immune response of the skin in the event of an allergy, for example.

Reconstructed skin can be obtained by using a culture device to culture human-adult keratinocytes on a collagen base, under conditions that enable them to differentiate and to reconstruct an epidermis provided with a functional corneum. The keratinocytes can be subjected to a stage of culture while immersed on a collagen support, and can then be kept immersed over a time period that makes it possible to obtain differentiation of the epidermis and the formation of a corneum.

Reconstructed skin is sold under the trade name Episkin®, for example.

In order, in particular, to enable new substances for treating the skin and/or for coming into contact therewith to be developed, it is necessary to make it easier to understand the biological, chemical, or physical characteristics of the skin.

There also exists a need to improve and/or to facilitate testing the safety and/or effectiveness of such substances.

In a first of its aspects, the invention provides a device comprising:
a sample of reconstructed tissue, and in particular of reconstructed skin; and
a detection system that is at least partially in contact with the sample and/or with a device serving to culture said sample, said detection system making it possible to measure the influence of a physical, biological, and/or chemical stress on at least a fraction of the sample, or vice-versa.

In one exemplary embodiment, said stress is an electrical, thermal, optical, chemical and/or biological stress.

In another exemplary embodiment, said detection system includes a sensor in contact with said sample.

In an embodiment of the invention, the detection system is configured to measure the influence of a physical, biological, and/or chemical stress on at least a fraction of the sample.

In another embodiment of the invention, the detection system is configured to measure the influence of at least a fraction of the sample on a physical, biological, and/or chemical stress.

The detection system may include a sensor and/or stress-imparting means in contact with the sample and/or in contact with the device serving to culture it.

Throughout the description and in the claims, the term “sensor” should not be understood in a narrow sense, but encompasses a variety of components that, on their own or in association with other means, are capable of supplying a useful signal in response to a physical, chemical, and/or biological stress being imparted to the sample. In particular, the detection system may include a sensor that is secured to the sample, while said sample is viable.

The expression “stress-imparting means” should likewise be understood broadly, and encompasses components that are, for example, capable of modifying at least one physical, chemical, and/or biological parameter of the sample, e.g. of generating a physical stress, in particular electrical, thermal, optical, mechanical, chemical, and/or biological, e.g. by releasing a chemical and/or biological entity.

The detection system may include various sensors, e.g.:
a sensor for detecting amino acids; vitamins; enzymes, in particular glucose dehydrogenase enzymes, glucamate dehydrogenase enzymes, and/or NadH oxidase enzymes; acethylcholine; and/or oxygenated water;
a sensor for detecting partial pressure of at least one gas, in particular a pₒ₂, p_{co2} sensor;
a sensor that is sensitive to the concentration of at least one ionic species, in particular a pH sensor;
a biological-tissue sensor;
at least one electrode in contact with the sample, said electrode possibly including, where appropriate, a material on its surface that is inert relative to the sample, in particular a passivated material;
one or more sensors and/or optical stress-imparting means, e.g. at least one of: an optrode; a photodiode; a phototransistor; a photoconductor; a laser; an optical fiber; and/or a bundle of optical fibers in contact with the reconstructed-skin sample; in particular a bundle of optical fibers or other means arranged to image a fraction of the sample and/or to analyze and/or to locate a chemical or biological entity in said sample;
a temperature sensor; and
a heating or cooling source enabling the sample to be heated and/or cooled, e.g. a Peltier-effect component, this list not being limiting.

In an embodiment of the invention, the detection system includes at least a source emitting UVa radiation and/or UVb radiation, and an optical sensor that is sensitive to ultraviolet radiation, in particular to UVa radiation and/or to UVb radiation.

Such a detection system can be useful, in particular so as to ascertain how much ultraviolet radiation is absorbed by the various layers of the sample, and so as to test new sun screens, for example.

In another embodiment of the invention, the detection system includes a light source enabling the sample to be exposed to light having a wavelength that is selected so as to excite a fluorescent marker, and a sensor that is sensitive to the wavelength of the light emitted by the fluorescent marker.

The reconstructed skin may comprise an epidermis containing keratinocytes. The reconstructed skin may optionally include a corneum. The reconstructed skin may contain melanocytes and/or Langerhans cells. The reconstructed skin preferably comprises both a dermis and an epidermis.

As indicated above, at least part of the detection system may be in contact with the sample.

By way of example, for a reconstructed-skin sample, at least part of the detection system may be positioned inside the epidermis, and in particular said system may include a sensor positioned, at least in part, inside the epidermis, e.g. below the corneum, in said corneum, or at the interface between the corneum and the keratinocytes.

Alternatively, at least part of the detection system may be positioned between the dermis and the epidermis, and in particular said system may include a sensor positioned between the dermis and the epidermis.

At least part of the detection system may be positioned inside the dermis, and in particular the system may include a sensor positioned inside the dermis.

At least part of the detection system may be positioned above or below the sample, and in particular the system may include a sensor positioned above or below the sample. When the detection system is positioned, at least in part, above the skin sample, the detection system may serve as a support for culturing the tissue cells.

Also, the detection system need not be in contact with the sample, being solely in contact with the culture device. The detection system may thus include a sensor that is separated from the sample solely by a layer of air, in particular when the sensor is an optical sensor disposed above the sample. The detection system may also include a sensor that is separated from the sample by at least one wall that does not belong to the detection system, in particular a wall belonging to the culture device.

The sample may present a greater or lesser area, e.g. lying in the range about 0.3 square centimeters (cm²) to about 1.35 cm², and in particular in the range about 0.38 cm² to about 1.12 cm².

The culture device may include a basket in which the sample is disposed, and a well to which the basket can be fastened in removable manner. The term “basket” should not be understood in a restrictive sense, and encompasses any sample support that is disposed, at least in part, in the well.

Where appropriate, the detection system may include a sensor that is disposed in at least one of the following locations: below the well; at the bottom of the well; in the thickness of a wall of the well, in particular the bottom wall of the well; below the basket; at the bottom of the basket; and/or in the thickness of a wall of the basket, in particular the bottom wall of the basket. The detection system may also include a sensor that is secured to a support fastened to one of: the well; and the basket. The support may be configured to be removable, where appropriate.

The detection system may include means for processing signals coming from a sensor and/or an interface for connection to a micro-computer.

In another of its aspects, the invention also provides, a method of measuring at least one physical, chemical, and/or biological parameter of at least one reconstructed-tissue sample, e.g. a reconstructed-skin sample, using a device as defined above, said method comprising the following steps:
exposing the sample to at least one physical, chemical, and/or biological stress; and
using the detection system to measure at least one physical, chemical, and/or biological parameter of the sample during and/or after said stress.

Before the sample is exposed to the stress, the value of the parameter may be measured. By way of example, said parameter may be the concentration of at least one chemical and/or biological entity which may be selected from: amino acids; vitamins; acethylcholine; oxygenated water; O₂, CO₂, H⁺; and/or enzymes, in particular glucose dehydrogenase enzymes, glucamate dehydrogenase enzymes, and/or NadH oxidase enzymes, this list not being limiting.

The stress may include exposing the sample to light radiation, in particular radiation comprising UVa radiation and/or UVb radiation. In this event, it is possible, for example, to measure the concentration of a chemical or a biological species whose presence is associated with light radiation, e.g. free radicals.

The skin may also be exposed to stress that is not optical, but that is thermal and/or mechanical.

The stress may include, or may be preceded by, applying a substance to the sample, in particular a cosmetic or a care product, e.g. a sun screen.

At least two measurements of said parameter may be taken, separated by a time interval, and they may possibly be compared, so as to determine the change in said parameter during stress.

In another of its aspects, the invention also provides a method of measuring the influence of at least one sample of reconstructed tissue of a device as defined above, on a physical, chemical, and/or biological stress parameter, said method comprising the following steps:
disposing the reconstructed-tissue sample, e.g. a reconstructed-skin sample, in such a manner as to enable it to influence the physical, chemical, and/or biological stress; and
using the detection system to collect at least one item of information that is representative of the influence of the sample on said stress.

The stress may be physical stress. By way of example, the stress may include exposing the sample to light radiation, in particular radiation comprising UVa radiation and/or UVb radiation. It is thus possible to measure a magnitude that is representative of the absorption, by the sample, of at least a fraction of the spectrum of the light radiation.

The physical stress may include at least one mechanical action, e.g. sending at least one ultrasound wave onto the sample and/or exposing said sample to at least one electrical stimulation, e.g. so as to measure a mechanical parameter, in particular elasticity, or an electrical parameter, e.g. electrical conductivity.

Where appropriate, at least part of the detection system may serve as a support for culturing the reconstructed tissue. The detection system may thus be present from the start of culturing the tissue, in particular skin. The detection system may possibly receive a surface treatment in order to passivate it so that it does not hinder the growth of the cells and does not damage said cells.

In a variant, the detection system may be implanted. while the tissue is being cultured, or it may be implanted in tissue that is completely reconstructed and viable. By way of example, at least part of the detection system may be implanted in the reconstructed tissue only while said system is being used, for example. In particular, the implantation of the detection system may take place between two measurements of a given parameter, for example.

The dimensions of the detection system may be adapted to enable it to be implanted in the sample, without substantially destroying said sample.

In another of its aspects, the invention also provides, a method of measuring at least one physical, chemical, and/or biological parameter of a sample of reconstructed tissue, in particular of reconstructed skin, in which the sample is subjected to a physical, chemical, and/or biological stress, and in which at least one measurement is taken of the parameter in the presence of the stress.

In one exemplary embodiment, said stress is an electrical, thermal, optical, chemical and/or biological stress.

By way of example, it is possible firstly to expose the sample to light radiation, and secondly to measure a magnitude that is representative of the absorption of said light radiation, by the sample.

The invention can be better understood on reading the following detailed description of non-limiting embodiments thereof, and on examining the accompanying drawings, in which:

FIG. 1 is a diagrammatic axial section of a known culture device for culturing a sample of reconstructed skin;

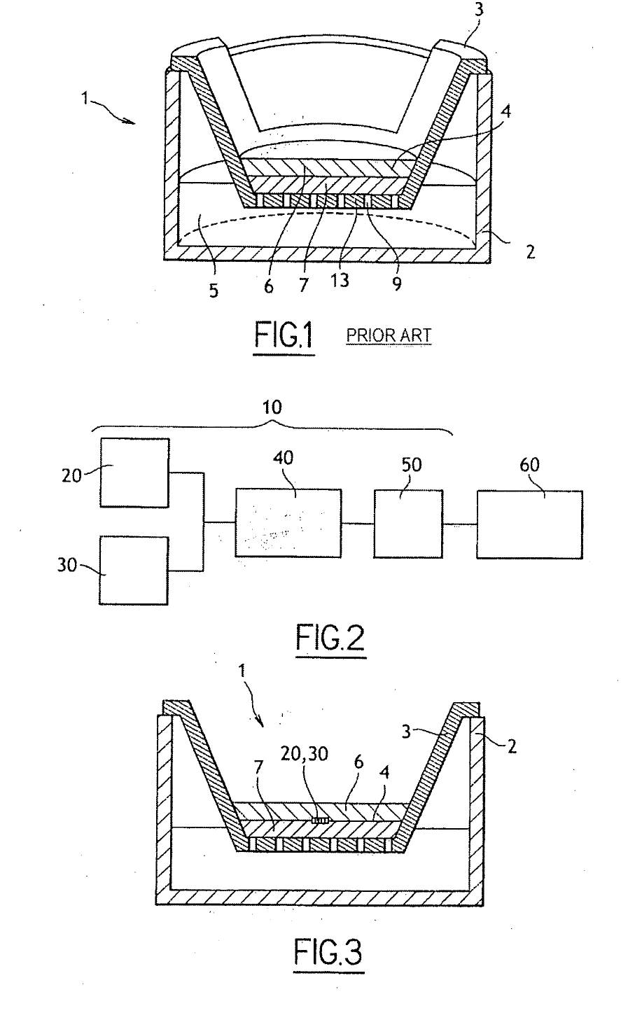

FIG. 2 is a block diagram of an example of a detection system; and

FIGS. 3 to 15 are diagrammatic and fragmentary axial sections showing various ways in which the detection system can be disposed relative to the reconstructed-skin sample and/or to the culture device.

FIG. 1 shows a known device for culturing a reconstructed-skin sample 4, also sometimes referred to as a skin equivalent, said device comprising a well 2 receiving a basket 3 carrying the sample 4.

The well 2 can form part of a plate having a plurality of wells, e.g. at least ten.

In the embodiment shown, the well 2 is partially filled with a solution 5, e.g. a nutrient medium.

The sample 4 can comprise an epidermis 6 and a dermis 7, said dermis comprising collagen, for example.

In the embodiment described, the basket 3 includes a bottom wall 13 supporting the skin sample 4. The bottom wall 13 has through perforations 9.

By way of example, the reconstructed-skin sample is cultured in accordance with one of the methods described in EP 0 789 074 B1, EP 0 789 074 A1, U.S. Pat. No. 6,079,415, and U.S. Pat. No. 6,660,522.

In accordance with the invention, a detection system 10 is associated with the sample 4.

FIG. 2 shows the possibility of the detection system 10 including a sensor 20 and/or a stress-imparting means 30, and means 40 for processing signals delivered by the sensor(s) 20, and for controlling the one or more stress-imparting means 30.

By way of example, the above-mentioned means 40 comprise specialized electronic circuits that are adapted to the nature(s) of the sensor(s) and/or the stress-imparting means 30.

The detection system 10 can include an integrated interface 50, making it possible to exchange data with a processor unit 60, which is constituted by a micro-computer, for example. By way of example, the interface 50 is an analog-to-digital interface, a series or parallel interface, a USB interface, or any other interface that makes it possible to exchange data with the micro-computer 60.

The detection system 10 includes one or more sensors 20 and/or stress-imparting means 30 disposed in contact with the skin sample 4 or with the culture device 2, 3.

For the purpose of clarity of the drawing, the sensor 20 and stress-imparting means 30 are represented diagrammatically in the figures by rectangles. It goes without saying that their shapes and dimensions can be various, and in particular can be adapted to be incorporated in the sample without substantially damaging said sample.

In addition, single rectangles 20 and 30 are shown, but the sensor 20 and the stress-imparting means 30 can each comprise a plurality of distinct components that are distributed in the skin sample and/or in or on the culture device 2, 3.

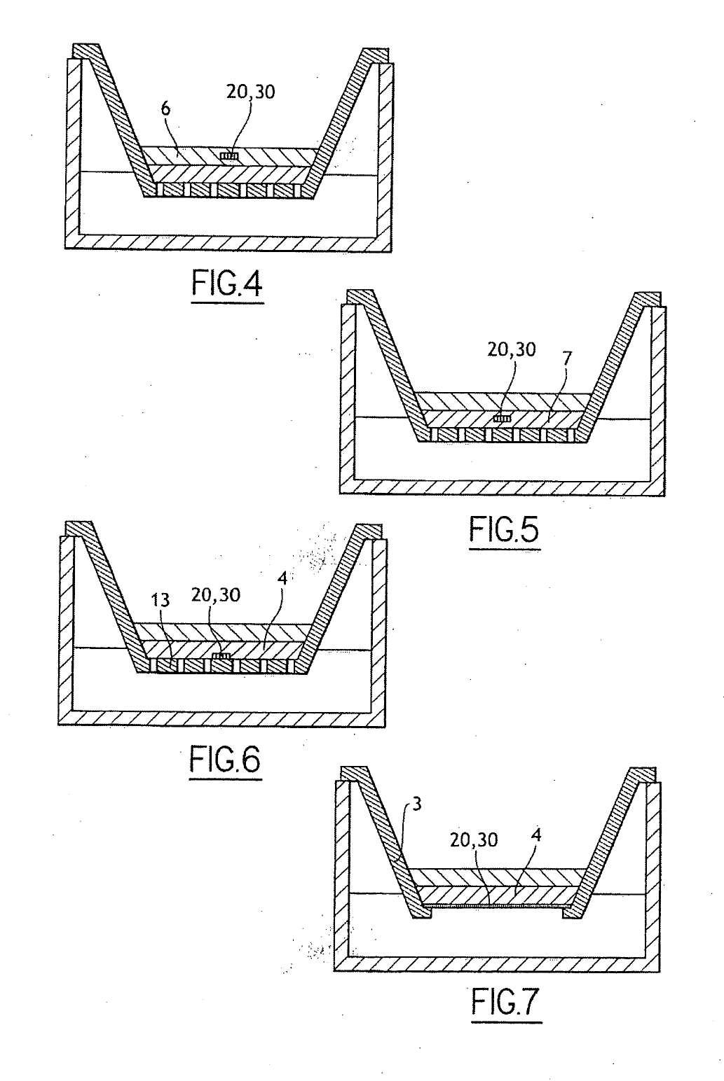

In the embodiment in FIG. 3, the sensor 20 and/or the stress-imparting means 30 is/are disposed, at least in part, between the dermis 7 and the epidermis 6; in the embodiment in FIG. 4, the sensor 20 and/or the stress-imparting means 30 is/are disposed, at least in part, inside the epidermis 6, e.g. in the corneum, in the keratinocytes, or at the interface between the corneum and the keratinocytes; and in the embodiment in FIG. 5, the sensor 20 and/or the stress-imparting means 30 is/are disposed, at least in part, inside the dermis 7.

In the embodiments in FIGS. 6 and 7, the sensor 20 and/or the stress-imparting means 30 is/are disposed below the skin sample 4. In this event, at least part of the detection system 10 is fastened to the bottom wall 13 before the stage of culturing the skin.

In the embodiment in FIG. 7, the detection system 10 serves entirely to support the skin sample. In particular, it is possible, in this way, to make a device in which a sensor that is sensitive to UV radiation serves, possibly after passivation, as a support to a keratinocyte culture having a stratum corneum on its surface. When such a device is placed below a UV lamp or a sun simulator, it can be particularly useful for measuring a flux of UVa radiation and/or of UVb radiation inside the epidermis, thereby making it possible to characterize the protective effects of a treatment, for example.

When the detection system 10 includes a sensor 20 that is disposed as shown in FIGS. 3 to 7, the sensor 20 can, for example, make it possible to detect and/or to quantify at least one chemical and/or biological entity that is present in contact therewith or in the vicinity thereof, e.g. an optionally-dissolved gas, reducing agent, oxidizing agent, ion, enzyme, and/or amino acid.

By way of example, the sensor 20 can be an enzyme sensor or a biological-tissue sensor.

By way of example, the sensor 20 can serve to detect amino acids, vitamins, acethylcholine, oxygenated water, the partial pressure of oxygen, the partial pressure of CO₂, and/or enzymes, in particular glucose dehydrogenase, glucamate dehydrogenase, and/or NadH oxidase, this list not being limiting

The sensor 20 can also serve to measure the pH of a given ionic species.

The sensor 20 can include one or more electrodes, and in particular at least one electrode made out of a material that is inert relative to the reconstructed-skin sample, e.g. a rare metal such as platinum, with the electrode serving to measure an electric current or a voltage, for example.

The sensor 20 can also be an optical sensor that is sensitive to ultraviolet radiation, for example, as mentioned above. By way of example, the sensor 20 includes a component such as a photodiode, a phototransistor, or a photoconductor. It can also be connected to one or more optical fibers, each having one end in contact with the sample.

By way of example, the sensor 20 can include a bundle of optical fibers each having a first end that is in contact with the sample, so as to obtain an image at the other end, e.g. an image of the corneocytes or of other cells in the sample, or an image that makes it possible to locate and/or to quantify a fluorescent compound in the sample.

The sensor 20 can also include means that make it possible to analyze and/or to locate a chemical and/or biological entity, e.g. means that make it possible to quantify an analyte by stimulated electroluminescence, in particular by using a bundle of optical fibers as described in the publication “A. Chovin et al, Development of an ordered array . . . , Annal. Chem. 2004, 76, 357-364”

The sensor(s) 20 can be secured to the skin sample when the skin is viable, or it can be fitted to said skin sample prior to use, so as to measure at least one physical parameter.

By way of example, when the skin sample is secured to one or more sensors 20, it is possible to culture the skin sample in the presence of said sensor(s). By way of example, it is possible to deposit keratinocytes on the surface of the sensor, said sensor possibly initially being passivated and then supporting culturing, with the sensor and the culture being immersed in a culture medium.

Also, the detection system 10 need not be directly in contact with the skin sample 4.

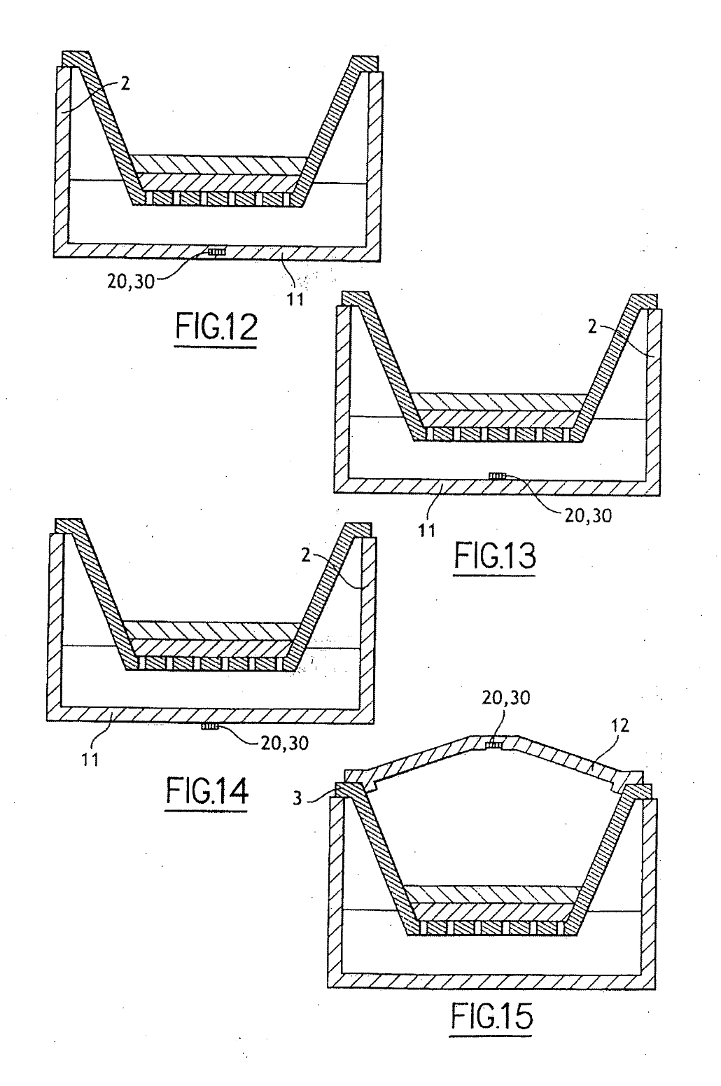

In the embodiment in FIG. 9, the detection system 10 includes a sensor 20 and/or stress-imparting means 30 disposed, at least in part, in the nutrient medium 5 by means of a support (not shown); in the embodiment in FIG. 10, the sensor 20 and/or the stress-imparting means 30 is/are disposed in a wall of the culture device, in this case in the thickness of the bottom wall 13 of the basket 3; in the embodiment in FIG. 11, the sensor 20 and/or the stress-imparting means 30 is/are disposed under the bottom wall 13 of the basket 3; in the embodiment in FIG. 12, the sensor 20 and/or the stress-imparting means 30 is/are disposed in a wall of the culture device, in this case in the thickness of the bottom wall 11 of the well 2; and in the embodiment in FIG. 13, the sensor 20 and/or the stress-imparting means 30 is/are disposed on the bottom wall of the well 2.

FIG. 14 shows the possibility of the detection system 10 including a sensor 20 and/or stress-imparting means 30 disposed, at least in part, under the bottom wall 11 of the well 2, and FIG. 15 shows the possibility of said detection system being secured to a support 12, which can be fastened in optionally-removable manner on the culture system, coming to rest against the top wall of the basket 3, for example.

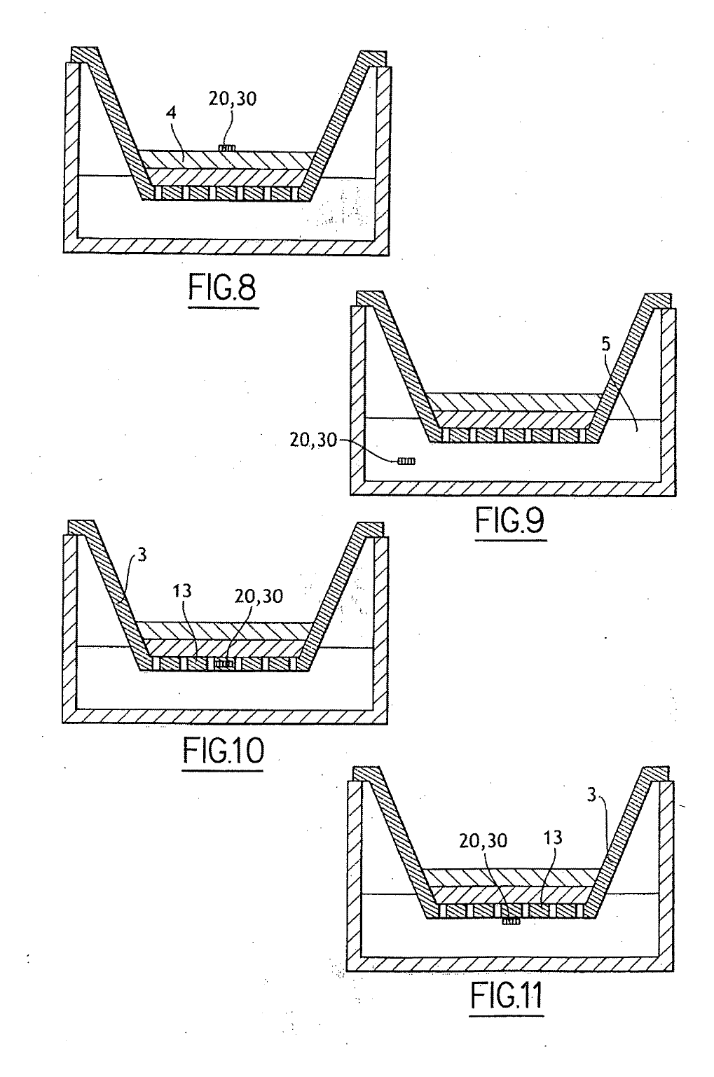

The examples in FIGS. 8 to 15 can be useful when the skin is exposed to light radiation, for example, and when the various thicknesses through which the light passes present optical properties that are compatible with the optical properties of the skin being measured through the thicknesses.

Thus, by way of example, the FIG. 11 embodiment corresponds to a sensor or to a light source 30 placed below a bottom wall 13 having light-absorbing characteristics that are known.

In variants that are not shown, the detection system includes more than one sensor or stress-imparting means, with each of the sensors or stress-imparting means being suitable, for example, for being disposed relative to the sample 4 in one of the above-mentioned configurations.

Naturally, the invention is not limited to the embodiments described above. In particular, sensors other than those described above can be used.

Where appropriate, all the wells of a common plate can be made in identical manner with identical sensors and/or stress-imparting means.

In a variant, at least two wells of a common plate can be made with sensors and/or stress-imparting means that are different.

The invention can be applied to reconstructed tissues other than skin.

Throughout the description, including in the claims, the expression “comprising a” should be understood as being synonymous with “comprising at least one”, unless specified to the contrary.

1-77. (canceled)

78. A device comprising:
a sample of reconstructed tissue; and
a detection system that is at least partially in contact with at least one of the sample and a device configured to culture said sample,
wherein the detection system is configured to measure one of:
an influence of at least one of a physical, biological, and chemical stress on at least a portion of the sample; and
an influence of at least a portion of the sample on at least one of a physical, biological, and chemical stress.

79. The device of claim 78, wherein the detection system is configured to measure the influence of at least one of a physical, biological, and chemical stress on at least a portion of the sample.

80. The device of claim 78, wherein the detection system is configured to measure the influence of at least a portion of the sample on at least one of a physical, biological, and chemical stress.

81. The device of claim 78, wherein the detection system comprises a sensor in contact with the sample.

82. The device of claim 78, wherein the detection system comprises a sensor in contact with a mechanism configured to culture the tissue.

83. The device of claim 78, wherein the detection system comprises a stress-imparting portion in contact with the sample.

84. The device of claim 78, wherein the detection system comprises a stress-imparting portion in contact with the device configured to culture the tissue.

85. The device of claim 78, wherein the detection system comprises a sensor for detecting at least one of amino acids, vitamins, acethylcholine, oxygenated water, and/or enzymes, glucose dehydrogenase enzymes, glutamate dehydrogenase enzymes, and NadH oxidase enzymes.

86. The device of claim 78, wherein the detection system includes a sensor for detecting partial pressure of at least one gas.

87. A device according to claim 86, wherein the sensor is a pₒ₂ or p_{co2} sensor.

88. The device of claim 78, wherein the detection system comprises a biological tissue sensor.

89. The device of claim 88, wherein the detection system comprises at least one electrode.

90. The device of claim 89, wherein the electrode is in contact with the sample.

91. The device of claim 89, wherein at least on its surface the electrode comprises a material that is inert relative to the sample.

92. The device of claim 91, wherein the material comprises a passivated material.

93. The device of claim 78, wherein the detection system comprises at least one of an optrode, an optical fiber, and a bundle of optical fibers in contact with the sample.

94. The device of claim 78, wherein the detection system is arranged to at least one of image a portion of the sample, analyze a chemical or biological entity in the sample, and locate a chemical or biological entity in the sample.

95. The device of claim 78, wherein the detection system comprises at least one optical sensor and light source.

96. The device of claim 95, wherein the light source is configured to emit at least one of UVa radiation and UVb radiation.

97. The device of claim 95, wherein the optical sensor is sensitive to at least one of UVa radiation and UVb radiation.

98. The device of claim 78, wherein the detection system comprises a light source enabling the sample to be exposed to light having a wavelength that is selected to excite a fluorescent marker, and wherein the detection system further comprises a sensor that is sensitive to a wavelength of light emitted by the fluorescent marker.

99. The device of claim 95, wherein the optical sensor includes at least one of a photodiode, a phototransistor, and a photoconductor.

100. The device of claim 78, wherein the detection system comprises a temperature sensor.

101. The device of claim 78, wherein the detection system comprises a laser.

102. The device of claim 78, wherein the detection system comprises at least one of a heating and a cooling source configured to alter a temperature of the sample.

103. The device of claim 78, wherein the detection system comprises a sensor that is sensitive to a concentration of at least one ionic species.

104. The device of claim 103, wherein the detection system comprises at least one pH sensor.

105. The device of claim 78, wherein the sample comprises a sample of reconstructed skin.

106. The device of claim 105, wherein the sample comprises an epidermis containing keratinocytes.

107. The device of claim 105, wherein the sample comprises a corneum.

108. The device of claim 105, wherein the sample does not comprise a corneum.

109. The device of claim 105, wherein the sample comprises melanocytes.

110. The device of claim 105, wherein the sample comprises Langerhans cells.

111. The device of claim 105, wherein the sample comprises both a dermis and an epidermis.

112. The device of claim 78, wherein at least part of the detection system is in contact with the sample.

113. The device of claim 78, wherein at least part of the detection system is positioned above the sample.

114. The device of claim 78, wherein the detection system comprises at least one of a sensor and a stress-imparting mechanism positioned above the sample.

115. The device of claim 78, wherein at least part of the detection system is positioned below the sample.

116. The device of claim 78, wherein the detection system comprises at least one of a sensor and a stress-imparting mechanism positioned below the sample.

117. The device of claim 106, wherein at least part of the detection system is positioned inside the epidermis.

118. The device of claim 117, wherein the detection system comprises at least one of a sensor and a stress-imparting mechanism positioned at least partially inside the epidermis.

119. The device of claim 111, wherein at least part of the detection system is positioned between the dermis and the epidermis.

120. The device of claim 119, wherein the detection system comprises at least one of a sensor and a stress-imparting mechanism positioned between the dermis and the epidermis.

121. The device of claim 111, wherein at least part of the detection system is positioned inside the dermis.

122. The device of claim 121, wherein the detection system comprises a sensor positioned inside the dermis.

123. The device of claim 78, wherein the sample presents a surface having an area ranging from about 0.3 cm² to about 1.35 cm².

124. The device of claim 123, wherein the sample presents a surface having an area ranging from about 0.38 cm² to about 1.12 cm².

125. The device of claim 78, further comprising a basket in which the sample is disposed.

126. The device of claim 125, further comprising a well to which the basket can be fastened in removable manner.

127. The device of claim 78, wherein the detection system comprises a means for processing signals coming from at least one sensor.

128. The device of claim 78, wherein the detection system comprises an interface configured for connection to a micro-computer.

129. A method of using the device of claim 78, the method comprising:
exposing the sample to at least one of a physical, chemical, and biological stress; and
measuring at least one of a physical, chemical, and biological parameter of the sample at least one of during and after the exposing.

130. The method of claim 129, wherein the tissue comprises reconstructed skin.

131. The method of claim 129, further comprising measuring the value of the parameter before the sample is exposed to the stress.

132. The method of claim 129, wherein the parameter is a concentration of at least one of a chemical and a biological entity.

133. The method of claim 132, wherein the at least one chemical and biological entity is chosen from at least one of amino acids, vitamins, acethylcholine, oxygenated water, O₂, CO₂, H⁺, enzymes, glucose dehydrogenase enzymes, glutamate dehydrogenase enzymes, and NadH oxidase enzymes.

134. The method of claim 129, wherein the stress is physical stress.

135. The method of claim 134, wherein exposing the sample to the stress comprises exposing the sample to light radiation comprising at least one of UVa radiation and UVb radiation.

136. The method of claim 135, further comprising measuring a magnitude that is representative of absorption by the sample of at least a fraction of the spectrum of the light radiation.

137. The method of claim 129, wherein exposing the sample to the stress comprises applying a substance to the sample.

138. The method of claim 137, wherein applying the substance to the sample comprises applying one of a cosmetic and a care product to the sample.

139. The method of claim 129, wherein exposing the sample to the stress comprises exposing the sample to thermal stress.

140. The method of claim 129, wherein exposing the sample to the stress comprises exposing the sample to mechanical stress.

141. The method of claim 129, wherein exposing the sample to the physical stress includes exposing the sample to at least one electrical stimulation.

142. The method of claim 129, wherein the measuring comprises measuring the at least one parameter at at least two time periods.

143. A method of measuring the influence of at least one sample of tissue of a device of claim 78, on at least one of a physical, chemical, and biological stress parameter, the method comprising:
disposing the sample in such a manner as to enable it to influence the at least one physical, chemical, and biological stress; and
collecting at least one item of information that is representative of the influence of the sample on said stress via the detection system.

144. The method of claim 143, wherein the tissue comprises reconstructed skin.

145. A method of manufacturing the device of claim 78, the method comprising:
culturing a reconstructed-tissue sample; and
supporting the reconstructed-tissue sample with at least part of the detection system during the culturing.

146. The method of claim 144, wherein the supporting comprises supporting the reconstructed-tissue sample with at least part of the detection system at a beginning of the culturing.

147. The method of claim 145, further comprising implanting the detection system in the sample while said sample is being cultured.

148. A method of manufacturing the device of claim 78, the method comprising:
implanting at least part of the detection system in tissue that is substantially completely reconstructed and viable.

149. The method of claim 148, wherein the implanting of at least part of the detection system occurs during use of the detection system.

150. The method of claim 149, wherein the implanting of at least part of the detection system occurs between two measurements taken by the detection system.

151. A method of measuring at least one of a physical, chemical, and biological parameter of a sample of reconstructed tissue, the method comprising:
subjecting the sample to at least one of a physical, chemical, and biological stress; and
taking at least one measurement of at least one physical, chemical, and biological parameter of the sample of reconstructed tissue during the subjecting of the sample to the at least one physical, chemical, and biological stress.
